(19) United States
(12) Patent Application Publication
(10) Pub. No.: US 2024/0425842 A1
(43) Pub. Date: Dec. 26, 2024

(54) METHOD FOR IMPROVING PLANT GROWTH WITH A TRNA SYNTHETASE GENE THAT ACTIVATES TOR

(71) Applicant: Wisconsin Alumni Research Foundation, Madison, WI (US)

(72) Inventor: Jacob Brunkard, Madison, WI (US)

(21) Appl. No.: 18/754,725

(22) Filed: Jun. 26, 2024

Related U.S. Application Data
(60) Provisional application No. 63/510,280, filed on Jun. 26, 2023.

(51) Int. Cl. C12N 9/00; C12N 15/82

(52) U.S. Cl. CPC C12N 9/93; C12N 15/8216; C12N 15/8262; C12Y 601/01022

(57) ABSTRACT

The present invention provides compositions and methods for increasing the growth, growth rate, and/or yield of a plant that lacks root nodules by engineering it to overexpress the protein asparaginyl-tRNA synthetase 1 (NARS1). Specifically, the present invention provides plants that are engineered to overexpress NARS1, seeds produced by said plants, and methods of generating and growing said plants.

### CROSS-REFERENCE TO RELATED APPLICATIONS

This application claims priority to U.S. Provisional Application No. 63/510,280, filed Jun. 26, 2023, the contents of which are incorporated by reference in their entireties.

### STATEMENT REGARDING FEDERALLY SPONSORED RESEARCH

This invention was made with government support under grant numbers OD023072 and GM145814 awarded by the National Institutes of Health. The government has certain rights in the invention.

### SEQUENCE LISTING STATEMENT

This application includes a sequence listing in XML format titled “960296.04519_ST26.xml”, which is 43,231 bytes in size and was created on Jun. 24, 2024. The sequence listing is electronically submitted with this application via Patent Center and is incorporated herein by reference in its entirety.

### BACKGROUND

Plants are an essential resource. We rely on them for food, water, medicine, clean air, habitat, shelter, and fuel. Climate change is expected to have a serious negative impact on our ability to grow plants. Genetic methods to improve crop plant growth rates and yields are urgently needed as demands for agricultural products (bioenergy/biomass, food, feedstock, etc.) increase while fertilizer resources and available arable land decrease. Accordingly, there is a need in the art for more efficient methods of growing plants.

### SUMMARY

The present invention provides compositions and methods for increasing the growth, growth rate, and/or yield of a plant that lacks root nodules by engineering it to overexpress the protein asparaginyl-tRNA synthetase 1 (NARS1).

In a first aspect, the present invention provides plants that lack root nodules and are engineered to overexpress a NARS1 protein.

In a second aspect, the present invention provides seeds produced by the plants described herein.

In a third aspect, the present invention provides methods of generating a plant that overexpresses NARS1. The methods comprise: (a) introducing a construct comprising a heterologous promoter operably linked to a polynucleotide encoding a NARS1 protein into a plant cell; and (b) growing the plant cell into a plant.

In a fourth aspect, the present invention provides methods of growing a plant that overexpresses NARS1. The methods comprise (a) planting a seed described herein; and (b) growing the seed into a plant.

### BRIEF DESCRIPTION OF THE DRAWINGS

FIGS. 1A-1B show a comparison of transgenic Arabidopsis thaliana plants that overexpress NARS1 to wild-type control plants after 7 days of growth. FIG. 1A shows representative images of the transgenic Arabidopsis thaliana seedlings (left) and the wild-type seedlings (right). FIG. 1B is two bar graphs showing the leaf width (left) and leaf length (right) of leaf 6 of wild-type control plants (“mock”), plants that overexpress NARS1 (“NRS1”), and plants that overexpress NARS2 (“NRS2”) after 7 days of growth. Leaf width and length were compared via t test (** p<10-5; * p<10-2; n≥24).

FIG. 2 shows a comparison of transgenic Arabidopsis thaliana plants that overexpress NARS1 to wild-type control plants after 4 weeks of growth. Representative images of Col-0 wild-type plants (top) and plants transformed with 35S:PRO:NRS1:pEG103 (bottom) are shown.

FIG. 3 is a bar graph comparing the leaf width and length of transgenic Arabidopsis thaliana plants that overexpress NARS1 to that of wild-type (Col-0) control plants. The length and width of leaf 6 was measured and compared via t test (** p<10-5; * p<10-2; n≥24).

FIG. 4 is an alignment of NARS1 protein sequences from 18 different plant species. The identity of each sequence is detailed in Table 1, below.

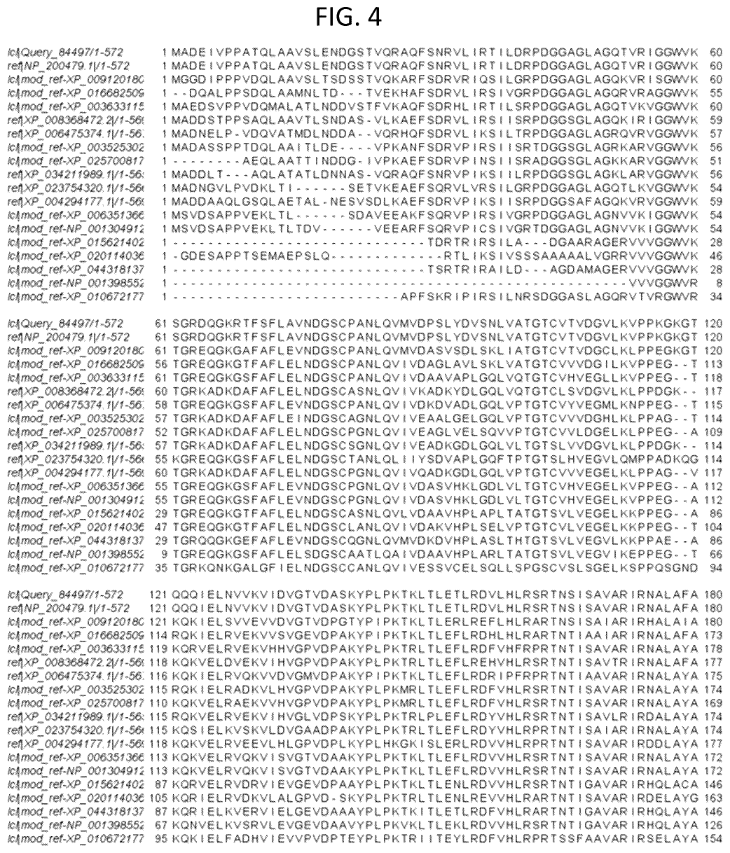

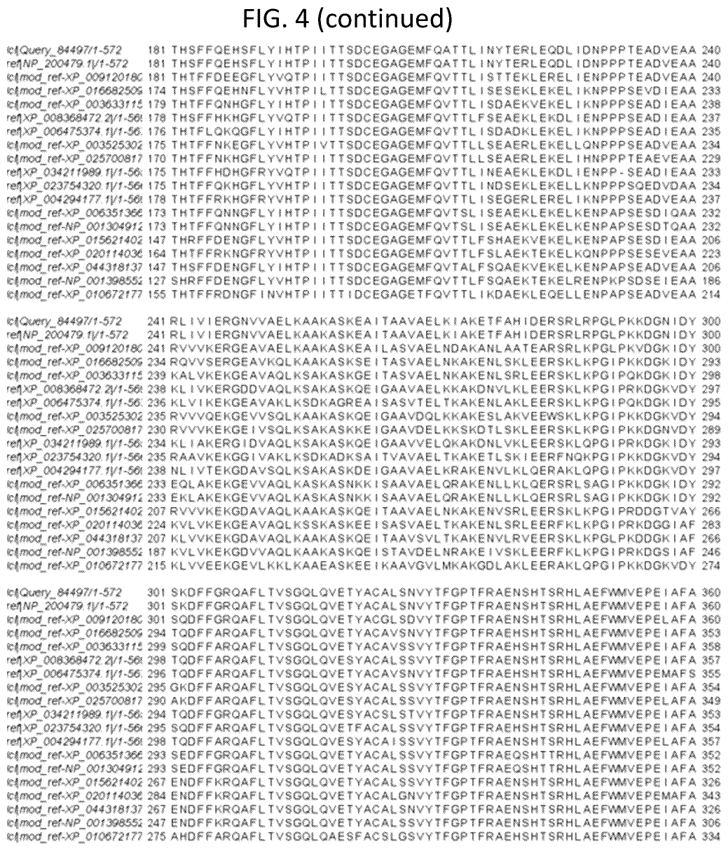

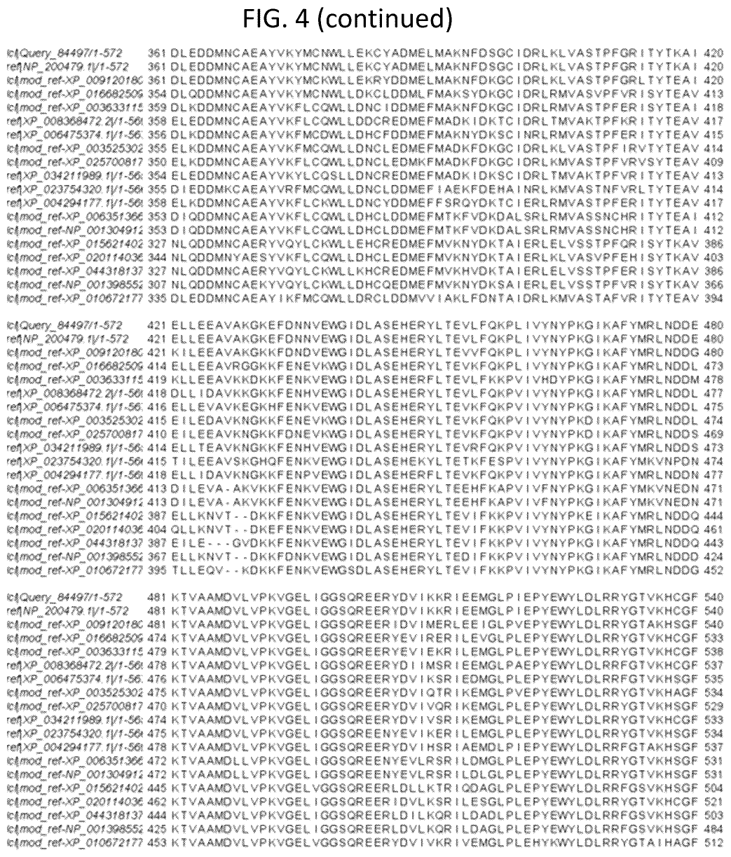

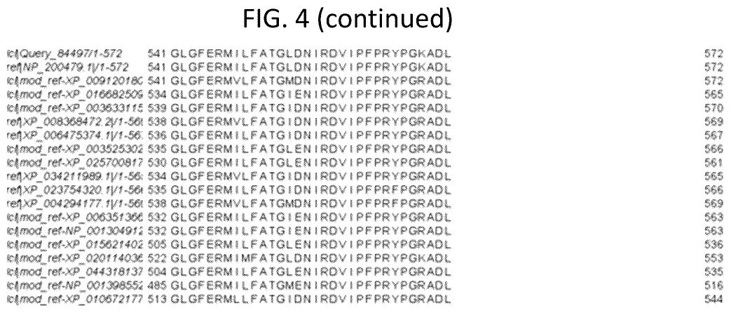

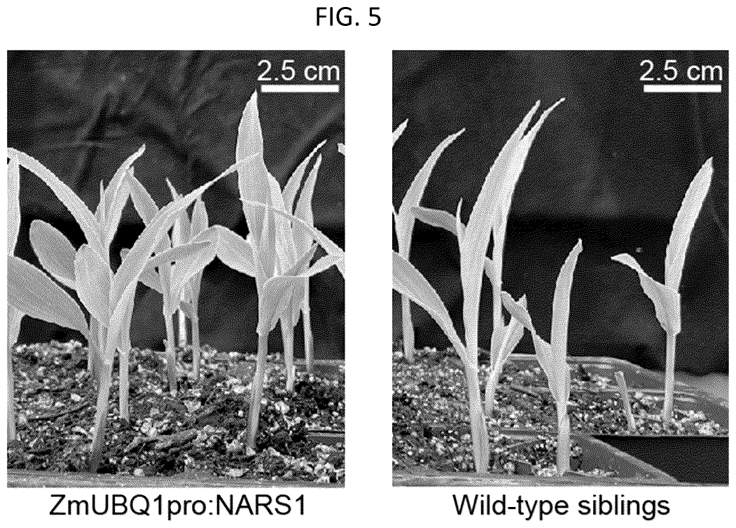

TABLE 1
NARS1 protein sequences aligned in FIG. 4

| Row in | SEQ ID | | |
|---|---|---|---|
| Alignment | NO: | Organism | Reference Genome |
| 1 | 2 | Query sequence | NR |
| 2 | 2 | Arabidopsis thaliana | NP_200479.1 |
| 3 | 3 | Brassica rapa | XP_009120180.1 |
| 4 | 4 | Gossypium hirsutum | XP_016682509.1 |
| 5 | 5 | Vitis vinifera | XP_003633115.1 |
| 6 | 6 | Malus domestica | XP_008368472.2 |
| 7 | 7 | Citrus sinensis | XP_006475374.1 |
| 8 | 8 | Glycine max | XP_003525302.1 |
| 9 | 9 | Arachis hypogaea | XP_025700817.1 |
| 10 | 10 | Prunus dulcis | XP_034211989.1 |
| 11 | 11 | Lactuca sativa | XP_023754320.1 |
| 12 | 12 | Fragaria vesca subsp. vesca | XP_004294177.1 |
| 13 | 13 | Solanum tuberosum | XP_006351366.1 |
| 14 | 14 | Solanum lycopersicum | NP_001304912.1 |
| 15 | 15 | Oryza sativaJaponica Group | XP_015621402.1 |
| 16 | 16 | Ananas comosus | XP_020114036.1 |
| 17 | 17 | Triticum aestivum | XP_044318137.1 |
| 18 | 18 | Zea mays | NP_001398552.1 |
| 19 | 19 | Beta vulgaris subsp. vulgaris | XP_010672177.2 |

FIG. 5 shows representative images of T1 generation (family A) transgenic maize plants that overexpress NARS1 (left) to wild-type control plants (right) two weeks after germination.

### DETAILED DESCRIPTION

The present invention provides plants that lack root nodules and are engineered to overexpress an asparaginyl-tRNA synthetase 1 (NARS1) protein, seeds produced by said plants, and methods of generating and growing said plants.

NARS1 is a cytosolic asparaginyl-tRNA synthetase, i.e., an enzyme that catalyzes the attachment of asparagine to its cognate tRNA for use in protein translation. In the Examples, the inventor demonstrates that both transgenic Arabidopsis thaliana plants (Example 1) and transgenic maize plants (Example 2) that overexpress the Arabidopsis thaliana NARS1 protein grow larger and faster than wild-type controls. These results suggest that NARS1 overexpression may be used to increase plant growth, plant growth rate, and/or yield.

In a previous study, researchers showed that overexpressing NARS1 in the legume Lotus corniculatus increased plant biomass and height in a single transgenic line (Yano et al., Plant Root 9:6-14, 2014). However, the researchers concluded that the increased biomass observed in this legume was due to increased nitrogen fixation due to an increased number of root nodules in these plants. Additionally, the same group later showed that overexpressing NARS1 in soybean increased plant height and bushiness in a single transgenic line (Arifin et al., Plant Biotechnol (Tokyo) 36 (4): 233-240, 2019). Thus, NARS1 overexpression was not expected to have a positive effect on growth or yield in plants lacking root nodules, and the inventor's discovery that increased NARS1 expression results in increased growth in Arabidopsis thaliana and maize was surprising.

### Plants:

In a first aspect, the present invention provides plants that lack root nodules and overexpress an asparaginyl-tRNA synthetase 1 (NARS1) protein. The plants may be engineered to overexpress NARS1 relative to a control plant using any method known to those of skill in the art.

The terms “protein,” “polypeptide,” and “peptide” are used interchangeably herein to refer to a polymer of amino acids, i.e., a series of amino acid residues connected by peptide bonds between the alpha-amino and carboxy groups of adjacent residues. Proteins may be modified (e.g., via acetylation, glycosylation, etc.) and may include amino acid analogs.

A used herein, a protein is “overexpressed” in a plant if it is expressed at higher levels than it is expressed in a control plant. For example, a plant that overexpresses a protein may express that protein at levels that are at least 25%, at least 50%, at least 2-times, at least 3-times, at least 4-times, at least 5-times, at least 6-times, at least 7-times, at least 8-times, at least 9-times, at least 10-times, at least 20-times, at least 30-times, at least 40-times, or at least 100-times higher than levels found in a control plant.

As used herein, a “control plant” is a comparable plant (i.e., a plant of the same species, variety, age, etc.) that was grown under substantially similar conditions but that was not engineered to overexpress a NARS1 protein. Plants that are grown in “substantially similar conditions” are grown in similar locations and soil conditions, are planted with similar timing, are subjected to similar treatments and abiotic stresses, and the like.

In some embodiments, the plant comprises a construct comprising a heterologous promoter operably linked to a polynucleotide encoding the NARS1 protein. As used herein, the term “construct” refers a to recombinant polynucleotide, i.e., a synthetic polynucleotide that was formed by combining at least two polynucleotide components from different sources. For example, a construct may comprise the coding region of one gene operably linked to a promoter that is (1) associated with another gene found within the same genome, (2) from the genome of a different species, or (3) synthetic. Constructs include plasmids, viral constructs, and transposon-based constructs. Constructs can be generated using conventional recombinant DNA methods. In some embodiments, the construct comprises multiple different NARS1-enocoding polynucleotides and/or multiple copies of a single NARS1-enocoding polynucleotide.

As used herein, the term “promoter” refers to a DNA sequence that defines where transcription of a polynucleotide begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from an endogenous or heterologous gene, may be composed of elements derived from multiple regulatory sequences found in nature, or may comprise synthetic DNA. A promoter is “operably linked” to a polynucleotide if the promoter is positioned such that it can affect transcription of said polynucleotide. The promoters used in the constructs of the present invention are “heterologous”, meaning that they are not naturally associated with the NARS1-enocoding polynucleotide to which they are operably linked.

In preferred embodiments, the promoter is a constitutive promoter. A “constitutive promoter” is a promoter that drives transcription of a polynucleotide in most cell types of an organism at most times. In Example 1, the inventor generates transgenic Arabidopsis thaliana plants that comprise a construct comprising a cauliflower mosaic virus (CaMV) 35S promoter/enhancer sequence (SEQ ID NO: 26), which comprises the CaMV 35S promoter of SEQ ID NO: 27, operably linked to a NARS1-encoding polynucleotide. In Example 2, the inventor generates transgenic maize plants that comprise a construct comprising the Zea mays ubiquitin 1 (UBQ1) promoter (SEQ ID NO: 24) operably linked to a NARS1-encoding polynucleotide. Both the CaMV 35S promoter and the maize UBQ1 promoter are considered strong constitutive promoters for transgene expression in plants. Thus, in certain embodiments, the promoter is a CaMV 35S promoter or a maize UBQ1 promoter.

In some embodiments, the promoter is an “inducible promoter,” i.e., a promoter that allows for controlled expression of a gene under particular conditions or in the presence of a particular molecule (e.g., tetracycline, dexamethasone). For example, the use of a starvation response promoter would allow for NARS1 overexpression only when nutrients are limiting, and the use of a drought-repressive promoter would allow for NARS1 overexpression only in the presence of a drought response.

Examples of other suitable promoters that may be used in the constructs of the present invention include, without limitation, other ubiquitin promoters, RuBisCO small subunit 1 (RbcS1) promoters, actin promoters, Agrobacterium octopine synthase (OCS) promoters, mannopine synthase (MAS) promoters, and Cestrum yellow leaf curling virus (CmYLCV) promoters.

The terms “polynucleotide,” “nucleic acid,” and “oligonucleotide” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages. The constructs of the present invention comprise a polynucleotide that encodes a NARS1 protein (i.e., a “NARS1-encoding polynucleotide”).

In the Examples, the inventor generated both transgenic Arabidopsis thaliana plants that comprise a construct encoding an endogenous NARS1 protein and transgenic maize plants that comprise a construct encoding a heterologous NARS1 protein (i.e., the same Arabidopsis thaliana NARS1 protein). Thus, the overexpressed NARS1 protein may be either an endogenous NARS1 protein (i.e., a NARS1 protein that is natively expressed by the plant) or a heterologous NARS1 protein (i.e., a NARS1 protein that is not natively expressed by the plant).

The plants of the present invention are engineered to overexpress a NARS1 protein. The term “engineered” is used herein to refer to plants that have been altered by the hand of man. Those of skill in the art are aware of multiple methods for engineering a plant to overexpress a particular protein. For example, a plant may be engineered to overexpress a NARS1 protein by introducing a NARS1-encoding polynucleotide into the plant using well-known recombinant or molecular biology techniques. In embodiments in which the NARS1 protein is an endogenous protein, the NARS1-encoding polynucleotide may comprise one or more extra copy of an endogenous polynucleotide (i.e., a polynucleotide that is natively found in the plant) that encodes NARS1. The endogenous NARS1-encoding polypeptide may optionally be altered to include synonymous mutations and/or additional sequences (e.g., adaptor sequences, a sequence encoding a reporter molecule or a protein tag), e.g., to increase protein expression or to increase the case of cloning or protein detection. In embodiments in which the NARS1 protein is a heterologous protein, the NARS1-encoding polynucleotide comprises a heterologous polynucleotide (i.e., a polynucleotide that is not natively found in the plant) that encodes NARS1. In some embodiments, the engineered plants are genetically modified. For example, in some embodiments, the NARS1-encoding polynucleotide is integrated into the genome of the plant (e.g., using an Agrobacterium vector, viral vector, nuclease, or transposase). In these embodiments, the NARS1-encoding polynucleotide may be either inserted randomly into the genome or inserted into a specific location (e.g., via homologous recombination). In other embodiments, the NARS1-encoding polynucleotide remains extrachromosomal (i.e., as part of an extrachromosomal plasmid).

Alternatively, a plant may be engineered to overexpress a NARS1 protein by upregulating the expression of an endogenous NARS1 gene. As used herein, a gene is “upregulated” if it has manipulated such that it is transcribed at higher levels than it would be in the absence of said manipulation. For example, an upregulated gene may be transcribed at levels that are at least 25%, at least 50%, at least 2-times, at least 3-times, at least 4-times, at least 5-times, at least 6-times, at least 7-times, at least 8-times, at least 9-times, at least 10-times, at least 20-times, at least 30-times, at least 40-times, or at least 100-times higher than the levels at which it would be transcribed in the absence of manipulation. In another alternative, the transcript may be made more stable such that additional NARS1 is generated as compared to a control plant. Upregulation can be accomplished by inserting a regulatory element into the genome such that it is operably linked to a target gene. Examples of suitable regulatory elements that can be used to upregulate a target gene include, without limitation, promoters, enhancers, and insulators. Alternatively, upregulation can be accomplished using CRISPR-mediated transcriptional activation (CRISPRa). In CRISPRa, a modified nuclease-dead form of Cas9 (dCas9) to which an activator domain has been attached is targeted to a target gene using guide RNAs and used to transcriptionally activate the target gene.

The plants of the present invention overexpress NARS1 in at least one tissue. The plants may express NARS1 in one, two, three, four, five, six, or more different tissues. For example, the plants may overexpress NARS1 in developing seeds to enhance seed size or yield, overexpress NARS1 in developing fruits to enhance fruit size or yield, overexpress NARS1 in leaves to potentially enhance photosynthesis, carbon sequestration, and/or leaf growth, or overexpress NARS1 in roots to enhance root growth and/or nutrient uptake. Alternatively, the plants may overexpress NARS1 in every tissue or substantially every tissue. Further, the plants may either overexpress NARS1 at one or more particular stages of development or may overexpress NARS1 throughout all developmental stages.

The term “plant” can refer to a plant at any stage of development or to any part of a plant, including a plant cutting, a plant cell, a plant cell culture, a plant organ, a plant tissue, a plant seed, or a plantlet. In some embodiments, the plant is selected from the group consisting of maize, tobacco, hemp, rice, canola, potato, wheat, cotton, and sugar beet. As is noted in the Examples, the inventor has generated both Arabidopsis thaliana and maize plants that overexpress the Arabidopsis thaliana NARS1 protein (SEQ ID NO: 2). Thus, in some embodiments, the plants are Arabidopsis thaliana or maize.

The plants of the present invention lack root nodules. A “root nodule” is a swelling on the root of a plant that allows the plant to house nitrogen-fixing rhizobia bacteria. (Note: Root nodules are primarily found in legumes, but are also found in Actinorhizal plants (e.g., alder and bayberry) and plants of the genus Parasponia.) Thus, the plants of the present invention are not capable of forming a symbiotic relationship with rhizobia that results in independent nitrogen fixation. In at least some embodiments, the plants are non-leguminous. The term “non-leguminous” refers to plants that do not belong to the Fabaceae family (i.e., plants that are not a legume).

The plants of the present invention may overexpress any plant-derived NARS1 protein. NARS1 is well conserved in plants, as is demonstrated in FIG. 4, which is an alignment of NARS1 protein sequences from 18 different plants (i.e., SEQ ID NOs: 2-19). Within the “superrosids” group of eudicots (which includes canola, cotton, grape, apple, citrus, soybean, peanut, almond, lettuce, and strawberry), NARS1 is about 70% identical at the amino acid level based on global alignments of the full-length protein (100% of NARS1 aligned). Within monocots (which include rice, wheat, maize, and banana), NARS1 is 70% identical at the amino acid level with local alignments (>90% of NARS1 aligned) or ˜63% identical based on an end-to-end alignment. The N-terminal 50 amino acids, which have no assigned or predicted function, do not align entirely. Within the “superasterids” group of eudicots (which includes sugar beets, potato, and tomato), NARS1 is >63% identical at the amino acid level based on global alignments of full-length NARS1 (100% of NARS1 aligned). As an outgroup, human cytosolic NARS1 is only 36% identical over 46% of the protein and 16.6% identical across the whole protein. Thus, in some embodiments, the NARS1 protein is at least 90% identical to an amino acid sequence selected from SEQ ID NOs: 2-19 (see Table 1). In the Examples, the inventor generated transgenic plants that overexpress the Arabidopsis thaliana NARS1 protein of SEQ ID NO: 2. Thus, in certain embodiments, the NARS1 protein is at least 90% identical to SEQ ID NO: 2. In some embodiments, the NARS1 protein comprises or consists of SEQ ID NO: 2. Seeds:

In a second aspect, the present invention provides seeds produced by the plants described herein. A “seed” is an embryonic plant enclosed in a protective outer covering. The seeds provided herein are engineered such that they will develop into plants that overexpress NARS1 in at least one tissue.

Methods of Generating Plants that Overexpress NARS1:

In a third aspect, the present invention provides methods of generating a plant that lacks root nodules and overexpresses NARS1. The methods comprise: (a) introducing a construct comprising a heterologous promoter operably linked to a polynucleotide encoding a NARS1 protein into a plant cell; and (b) growing the plant cell into a plant.

The term “plant cell” refers to any cell of a plant. A plant cell is the basic structural and functional unit of plants. A plant cell comprises a protoplast and a cell wall. A plant cell can be in the form of an isolated single cell, part of an aggregate of cells, or part of a higher order structure or plant. The plant cells used with the present invention are part of or are derived from plants that lack root nodules.

As used herein, “introducing” describes a process by which exogenous polynucleotides are introduced into a recipient cell. Suitable introduction methods include, without limitation, Agrobacterium-mediated transformation, transposition-based plant transformation, the floral dip method, bacteriophage or viral infection, electroporation, heat shock, lipofection, microinjection, vacuum-infiltration, and particle bombardment.

In the Examples, the inventor utilized Agrobacterium-mediated transformation to introduce NARS1-encoding polynucleotides into plants. Thus, in preferred embodiments, the polynucleotides are introduced via Agrobacterium-mediated transformation. In this method, the NARS1-encoding polynucleotide is delivered into plant cells as part of a binary Agrobacterium vector, in which it is flanked by two transfer DNA (T-DNA) border repeat sequences. Prior to transformation into plant cells, this binary vector is co-transformed into Agrobacterium tumefaciens along with a second vector that is referred to as a vir helper plasmid. The vir helper plasmid encodes components necessary for integration of the region flanked by the T-DNA border repeat sequences into the genome of plant cells. Thus, when the binary vector and the vir helper plasmid are both present in the same Agrobacterium cell, proteins encoded by the vir helper plasmid act in trans on the T-DNA border repeat sequences to mediate processing, secretion, and host genome integration of the intervening transgene. Genome insertion occurs without any significant bias with respect to insertion site sequence.

In the present methods, NARS1-encoding constructs are introduced into a plant cell. Suitable constructs and parts thereof (i.e., heterologous promoters and NARS1-encoding polynucleotides) for use in these methods are described above, in the section titled “Plants.”

As used herein, “growing” describes a process in which suitable conditions (i.e., light, soil, water, nutrients, temperature) for plant growth are established and maintained.

Any type of plant that lacks root nodules may be generated using the present methods. In some embodiments, the plant is selected from the group consisting of maize, tobacco, hemp, rice, canola, potato, wheat, cotton, and sugar beet. In some embodiments, the plant is Arabidopsis thaliana or maize.

Methods of Growing Plants that Overexpress NARS1:

In a fourth aspect, the present invention provides methods of growing a plant that lacks root nodules and overexpresses NARS1. The methods comprise (a) planting a seed described herein; and (b) growing the seed into a plant. As used herein, “planting” describes a process in which a seed in placed in soil or another suitable growth medium (e.g., peat moss, coconut coir, vermiculite, perlite, sand, polymer-based gels).

In the Examples, the inventor demonstrates that transgenic Arabidopsis thaliana and maize plants that overexpress NARS1 grow larger and faster than wild-type control plants. Thus, in some embodiments, the plant grown using the present methods has an increased growth rate and/or yield as compared to a control plant.

The term “growth rate” describes the rate at which the size of a plant increases. Growth rate can be assessed, for example, by measuring the leaf area, leaf width, leaf length, leaf number, stem length, rosette diameter, root length, seed number, seed weight, height, biomass, or volume of a plant over time.

The term “yield” describes the amount of harvestable (i.e., useful) material produced by a plant. Examples of harvestable plant materials include, without limitation, flowers, pollen, seedlings, tubers, leaves, stems, fruit, seeds, and roots.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents form part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

In the following example, the inventor demonstrates that transgenic Arabidopsis thaliana plants that overexpress NARS1 grow larger and faster than wild-type controls.

### BACKGROUND

Target of rapamycin (TOR) is a highly conserved serine/threonine protein kinase that coordinates growth and development with nutritional status in eukaryotes. TOR regulates key pathways such as nucleotide biosynthesis, ribosome biogenesis, and leaf initiation. TOR is well-studied in humans because is it dysregulated in many human diseases, including diabetes and cancer. In animals and fungi, many of the upstream mechanisms that activate TOR are known, but almost nothing is known about how TOR activity is regulated in plants.

Asparaginyl-tRNA synthetase 1 (NARS1; official names: EMB2755 and SYNC1; gene ID: At5g56680) was identified as a potential regulator of TOR in plants in a forward genetic screen for defective embryonic cell-cell (plasmodesmatal) trafficking that repeatedly identified mutants defective in TOR signaling (Brunkard et al., Proc. Natl. Acad. Sci. U.S.A 117 (9): 5049-5058, 2020; Kim et al., Development 129 (5): 1261-1272, 2002). NARS1 mutants have stunted growth and defective TOR signaling. RNA sequencing revealed that NARS1 mutants exhibit several signatures of TOR inactivation, including significant repression of ribosomal protein genes and induction of proteolytic and catabolism-related genes (Busche et al., Plant Cell 33 (5): 1615-1632, 2021; Xiong et al., Nature 496 (7444): 181-186, 2013). Further, NARS1 mutants and knockdowns also show significantly less phosphorylation of S6K-T449 (orthologous to human S6K-T389), a residue that is uniquely phosphorylated by TOR kinase in plants. Thus, NARS1 activates TOR in plants and represents the first proposed amino acid sensor in plants.

### Materials and Methods:

RNA was extracted from Arabidopsis thaliana Col-0 and used as a template for RT-PCR with SuperScript III and Phusion, following the manufacturer's instructions. The NARS1 coding sequence was amplified from this RNA using the forward primer caccATGGCTGATGAGATTGTG (SEQ ID NO: 20) and the reverse primer AAGATCAGCTTTTCCAGGATAGCG (SEQ ID NO: 21), which were synthesized by IDT DNA. The forward primer included a CACC overhang for directional cloning into D-TOPO pENTR vectors. The reverse primer was designed to exclude the final stop codon from the NARS1 coding sequence to allow translational readthrough to C-terminal epitope tags. The PCR product size was verified by agarose gel electrophoresis and was purified using a gel extraction kit (NEB Monarch kit).

The product was subcloned into pENTR using the D-TOPO pENTR kit (Invitrogen) and used to transform chemically competent E. coli cells (genotype DH10B) using kanamycin selection. Resistant colonies were screened for by colony PCR and positive transformants were used in minipreps to isolate plasmid (NEB Monarch kit). Purified plasmid was sequenced using Sanger sequencing. This plasmid was digested with EcoRV and recombined with pEarleyGate 103 using Gateway LR Clonase (for full plasmid information, see abrc.osu.edu/stocks/number/CD3-685 [abrc.osu.edu]). pEarleyGate 103 includes a C-terminal GFP tag and a CaMV 35S promoter. Insertion into pEarleyGate 103 was validated using Sanger sequencing. The resulting plasmid is referred to herein as 35S: PRO: NRS1: pEG103.

35S: PRO: NRS1: pEG103 was introduced into Agrobacterium tumefaciens (genotype GV3101) and used to transform Arabidopsis thaliana inflorescences following the standard floral dip protocol. Positive transformants were screened for herbicide resistance, NARS1 insertions were validated by PCR, and NARS1 overexpression was validated by fluorescence microscopy. Dwarf transgenic lines showed an insertion but no GFP fluorescence, indicating gene silencing, whereas large transgenic lines showed strong GFP fluorescence in leaves.

### Results:

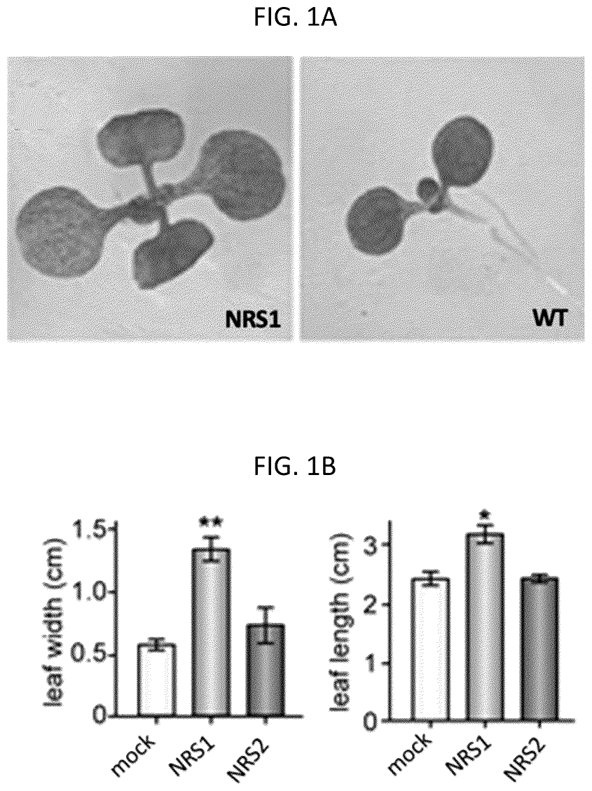

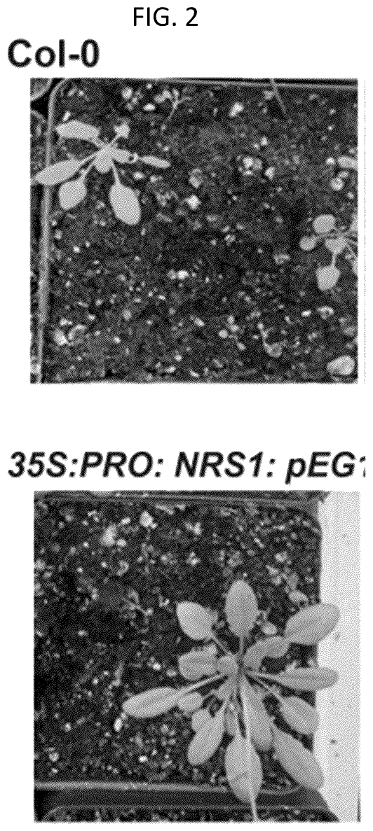

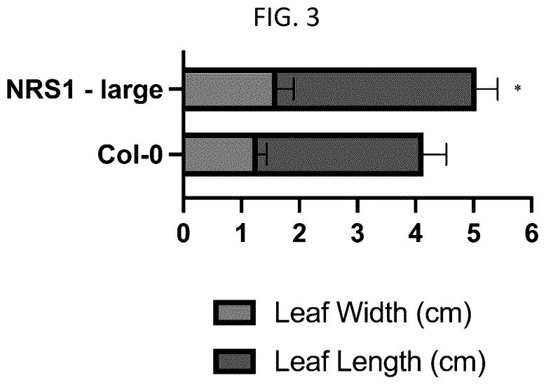

To test the effects of constitutively overexpressing NARS1 in plants, the Arabidopsis thaliana NARS1 coding sequence (SEQ ID NO: 1, which encodes the NARS1 protein of SEQ ID NO: 2) was cloned into pEarleyGate 103 and was used to transform Arabidopsis thaliana ecotype Col-O using an Agrobacterium vector (GV3101) and the floral dip method. Several stable transgenic Arabidopsis thaliana lines that overexpress NARS1 fused to a GFP reporter were generated. Consistently, NARS1 transgenic plants with visible GFP fluorescence (which indicates successful overexpression of the NARS1 protein) were found to grow larger and faster than wild-type controls (FIG. 1A). On average, NARS1 overexpression significantly increased the leaf width and length compared to wild-type controls (Col-0) (FIG. 2 and FIG. 3). This phenotype is similar to TOR overexpression, which causes increased organ and cell size. Yet, overexpressing another annotated asparaginyl tRNA synthetase protein, i.e., NARS2, had no effect on plant growth or TOR activity (FIG. 1B). This indicates that this effect is specific to NARS1 and not a general property of asparaginyl tRNA synthetases. These results suggest that NARS1 overexpression could be used in agriculture to improve plant growth and yield.

### Example 2

In the following example, the inventor demonstrates that transgenic maize plants that overexpress NARS1 grow larger and faster than wild-type controls.

### Materials and Methods:

### Cloning ZmUbi1_{PRO}:NARS1

The Arabidopsis thaliana NARS1 coding sequence was subcloned into a plasmid such that it was flanked by the promoter and terminator from the Zea mays ubiquitin 1 (UBQ1) gene to drive high, consistent levels of NARS1 expression throughout maize development. Specifically, an open reading frame (ORF) encoding the Arabidopsis thaliana NARS1 protein was synthesized de novo using GenSmart to optimize codons for expression in maize. Adaptors were added to the 5′ and 3′ ends of the ORF to facilitate GoldenGate cloning. The 5′ adaptor sequence is GGTCTCTA and the 3′ adaptor sequence is GCTTTGAGACC (SEQ ID NO: 22). Both adaptor sequences include Bsal recognition sites. The sequence of the resulting construct is provided as SEQ ID NO: 23.

This construct was subcloned, using a GoldenGate cloning strategy, into the T-DNA of a binary vector. The resulting vector includes the Zea mays Ubiquitin 1 (ZmUbi1) promoter (SEQ ID NO: 24), which includes a 5′ leader sequence with an intron, upstream of the NARS1 coding sequence. The resulting vector also includes the ZmUbi1 terminator (SEQ ID NO: 25), which includes a 3′ untranslated region, downstream of the NARS1 coding sequence. The resulting binary vector is referred to herein as ZmUbi1_{PRO}:NARS1.

### Maize Transformation

Maize cells of inbred genotype LH244 were transformed using Agrobacterium tumefaciens carrying the ZmUbi1_{PRO}:NARS1 binary vector, which also includes a gene that confers resistance to the herbicide glufosinate. Transformants were selected for glufosinate resistance and cultured under conditions to induce plant regeneration. Mature transformants were self-pollinated to generate T1 seeds for further analysis.

### Growth Assays

T1 seeds from two independently transformed parents were sown on wet potting soil and grown in a greenhouse in Madison, WI with ˜16 h daylength for two weeks. The transgenic plants were grown alongside (i.e., in the same flat) untransformed LH244 sibling plants. All seeds germinated at similar times. Two weeks after germination, plants were photographed and analyzed for differences in growth and vigor.

### Image Analysis

All images were analyzed using ImageJ.

### Results:

T1 ZmUbi1_{PRO}:NARS1 plants from two independent parents (family A and family B) were compared to wild-type sibling controls two weeks after germination in the greenhouse on potting soil mix (FIG. 5). Under these conditions, the average height of wild-type LH244 plants was 6.8 cm (n=7, standard deviation 3.4 cm), the average height of family A ZmUbi1_{PRO}:NARS1 plants was 8.9 cm (n=9, standard deviation 2.1 cm), and the average height of family B ZmUbi1_{PRO}:NARS1 plants was 8.6 cm (n=5, standard deviation 2.1 cm). Therefore, the ZmUbi1_{PRO}:NARS1 plants from family A were 30.6% taller than wild-type plants, and the ZmUbi1_{PRO}:NARS1 plants from family B were 26.6% taller than wild-type plants, on average. Transgenic ZmUbi1_{PRO}:NARS1 plants also exhibited less variability in height, with standard deviations that were 61.5% lower than the standard deviations observed for wild-type plants. Transgenic ZmUbi1_{PRO}:NARS1 plants from both families also qualitatively appeared to have larger, greener, and more vigorous shoots than the wild-type plants.

What is claimed:

1. A plant that lacks root nodules and is engineered to overexpress an asparaginyl-tRNA synthetase 1 (NARS1) protein as compared to a control plant.

2. The plant of claim 1, wherein the plant comprises a construct comprising a heterologous promoter operably linked to a polynucleotide encoding the NARS1 protein.

3. The plant of claim 2, wherein the heterologous promoter is a constitutive promoter.

4. The plant of claim 3, wherein the constitutive promoter is a cauliflower mosaic virus (CaMV) 35S promoter or a ubiquitin promoter.

5. The plant of claim 1, wherein the plant is a non-leguminous plant.

6. The plant of claim 1, wherein the plant is selected from the group consisting of maize, tobacco, hemp, rice, canola, potato, wheat, cotton, and sugar beet.

7. The plant of claim 1, wherein the plant is Arabidopsis thaliana or maize.

8. The plant of claim 1, wherein the NARS1 protein is an endogenous NARS1 protein.

9. The plant of claim 8, wherein expression of an endogenous gene encoding the NARS1 protein is upregulated in the plant.

10. The plant of claim 1, wherein the NARS1 protein is a heterologous NARS1 protein.

11. The plant of claim 1, wherein the NARS1 protein is at least 90% identical to an amino acid sequence selected from SEQ ID NOs: 2-19.

12. The plant of claim 1, wherein the NARS1 protein is overexpressed in at least one tissue selected from roots, leaves, fruit, or seeds of the plant.

13. A seed produced by the plant of claim 1.

14. A method of generating a plant that overexpresses a NARS1 protein, the method comprising:
a) introducing a construct comprising a heterologous promoter operably linked to a polynucleotide encoding the NARS1 protein into a plant cell from a plant that lacks root nodules; and
b) growing the plant cell into the plant that overexpresses the NARS1 protein.

15. The method of claim 14, wherein the plant is selected from the group consisting of Arabidopsis thaliana, maize, tobacco, hemp, rice, canola, potato, wheat, cotton, and sugar beet.

16. The method of claim 14, wherein the NARS1 protein is at least 90% identical to an amino acid sequence selected from SEQ ID NOs: 2-19.

17. A plant or seed produced by the method of claim 14.

18. A method of growing a plant that overexpresses NARS1, the method comprising:
a) planting the seed of claim 13; and
b) growing the seed into a plant that overexpresses NARS1.

19. The method of claim 18, wherein the plant has an increased growth rate as compared to a control plant.

20. The method of claim 18, wherein the plant has an increased yield as compared to a control plant.


SEQUENCE LISTING (27 sequences)

SEQ ID NO: 1 - DNA, 1716 bp, Arabidopsis thaliana
  source 1..1716: mol_type = genomic DNA
atggctgatgagattgtgccgccggcgactcaattagccgccgtatccttagaaaacgatggatctacggtgcaaagagctcaattctctaaccgggttttgattcgaacgattttggatcgacctgacggcggagctggactcgccggtcaaacggttcgaatcggtggttgggtaaaatccggaagagatcaagggaaaagaacgttttctttccttgcggttaacgatggatcttgtccagcgaatcttcaggttatggtggatccgtctctctatgatgtctctaacttggttgctacgggaacttgtgtgactgttgatggagttttgaaggttccaccgaaaggtaaaggtacgcagcagcagattgagcttaacgttgtgaaggtgattgatgttggaacggtggatgcgtctaagtatccacttcctaagactaagttgactcttgagactttgagagatgtgcttcatcttcgttctagaactaactcgatctcagctgttgcaagaattcgtaatgctcttgcttttgcgactcacagtttctttcaagagcatagtttcctttacattcacactcctatcatcacgacaagtgattgtgaaggtgctggtgaaatgttccaagcaacaactttgatcaattacactgagaggttggagcaggatctcattgataaccctccgcctactgaggctgatgttgaagctgcgaggctcattgtcattgagagaggtaatgttgtagcggaactgaaagctgccaaagcaagcaaggaagcgatcactgctgctgttgctgaacttaaaatagcaaaggagacttttgctcatattgatgagaggtcgaggcttagacctggactacctaagaaagatggaaacattgattattccaaggatttctttggtcgtcaagctttcttgacagtttccggtcaattacaagtggagacctatgcttgtgctcttagcaatgtgtatacatttggcccgactttccgggcagagaactctcacacttcaaggcatcttgctgaattctggatggttgagcctgagattgcttttgcagatctagaggacgacatgaactgtgcagaggcgtatgtgaaatacatgtgcaactggttgcttgagaaatgttacgctgacatggaacttatggctaagaatttcgactcaggatgcattgacaggctaaaattggttgcctctactccgtttgggcgtataacatacactaaagcaatagagctacttgaggaagctgtggctaaaggaaaggaatttgataacaatgtggagtggggaatcgacttagcctcggagcatgaaagatacttgacagaggttctgtttcaaaagccacttattgtctataactacccgaaaggaatcaaagctttttacatgagacttaacgatgatgagaagacagttgctgccatggatgtcctcgttccaaaggttggagaactcattggtggaagccaaagggaagaacgatatgatgtcatcaaaaagaggattgaggagatgggtcttccaatagagccatatgagtggtacctagacttgcgccgttatggaacagtgaagcattgtgggtttggacttggcttcgaacgtatgattctcttcgctactggacttgataacatcagagacgttattcctttccctcgctatcctggaaaagctgatctt

SEQ ID NO: 2 - protein, 572 aa, Arabidopsis thaliana
MADEIVPPATQLAAVSLENDGSTVQRAQFSNRVLIRTILDRPDGGAGLAGQTVRIGGWVKSGRDQGKRTFSFLAVNDGSCPANLQVMVDPSLYDVSNLVATGTCVTVDGVLKVPPKGKGTQQQIELNVVKVIDVGTVDASKYPLPKTKLTLETLRDVLHLRSRTNSISAVARIRNALAFATHSFFQEHSFLYIHTPIITTSDCEGAGEMFQATTLINYTERLEQDLIDNPPPTEADVEAARLIVIERGNVVAELKAAKASKEAITAAVAELKIAKETFAHIDERSRLRPGLPKKDGNIDYSKDFFGRQAFLTVSGQLQVETYACALSNVYTFGPTFRAENSHTSRHLAEFWMVEPEIAFADLEDDMNCAEAYVKYMCNWLLEKCYADMELMAKNFDSGCIDRLKLVASTPFGRITYTKAIELLEEAVAKGKEFDNNVEWGIDLASEHERYLTEVLFQKPLIVYNYPKGIKAFYMRLNDDEKTVAAMDVLVPKVGELIGGSQREERYDVIKKRIEEMGLPIEPYEWYLDLRRYGTVKHCGFGLGFERMILFATGLDNIRDVIPFPRYPGKADL

SEQ ID NO: 3 - protein, 572 aa, Brassica rapa
MGGDIPPPVDQLAAVSLTSDSSTVQKARFSDRVRIQSILGRPDGGAGLAGQKVRISGWVKTGREQGKGAFAFLEVNDGSCPANLQVMVDASVSDLSKLIATGTCVTVDGCLKLPPEGKGTKQKIELSVVEVVDVGTVDPGTYPIPKTKLTLERLREFLHLRARTNSISAIARIRHALAIATHTFFDEEGFLYVQTPIITTSDCEGAGEMFQVTTLISTTEKLERELIENPPPTEADVEAARVVVKERGEAVAELKAAKASKEAILASVAELNDAKANLAATEARSRLKPGLPKVDGKIDYSQDFFGRQAFLTVSGQLQVETYACGLSDVYTFGPTFRAENSHTSRHLAEFWMVEPELAFADLEDDMNCAEAYVKYMCKWLLEKRYDDMELMAKNFDKGCIDRLKLVASTPFGRLTYTEAIKILEEAVAKGKKFDNDVEWGIDLASEHERYLTEVVFQKPLIVYNYPKGIKAFYMRLNDDGKTVAAMDVLVPKVGELIGGSQREERIDVIMERLEEIGLPVEPYEWYLDLRRYGTAKHSGFGLGFERMVLFATGMDNIRDVIPFPRYPGRADL

SEQ ID NO: 4 - protein, 565 aa, Gossypium hirsutum
DQALPPSDQLAAMNLTDTVEKHAFSDRVLIRSIVGRPDGGAGLAGQRVRAGGWVKTGREQGKGTFAFLELNDGSCPANLQVIVDAGLAVLSKLVATGTCVVVDGILKVPPEGTRQKIELRVEKVVSVGEVDPAKYPIPKTKLTLEFLRDHLHLRARTNTIAAIARIRNALAFATHSFFQEHNFLYVHTPILTTSDCEGAGEMFQVTTLISESEKLEKELIKNPPPSEVDIEAARQVVSERGEAVKQLKAAKASKSEITASVAELNKAKENLSKLEERSKLKPGIPKKDGKIDYTQDFFARQAFLTVSGQLQVETYACAVSNVYTFGPTFRAEHSHTSRHLAEFWMVEPEIAFADLQDDMNCAEAYVKYMCNWLLDKCLDDMLFMAKSYDKGCIDRLRMVASVPFVRISYTEAVELLEEAVRGGKKFENEVKWGIDLASEHERYLTEVKFQKPLIVYNYPKGIKAFYMRLNDDLKTVAAMDVLVPKVGELIGGSQREERYEVIRERILEVGLPLEPYEWYLDLRRYGTVKHCGFGLGFERMILFATGIENIRDVIPFPRYPGRADL

SEQ ID NO: 5 - protein, 570 aa, Vitis vinifera
MAEDSVPPVDQMALATLNDDVSTFVKAQFSDRHLIRTILSRPDGGAGLAGQTVKVGGWVKTGREQGKGSFAFLELNDGSCPANLQVIVDAAVAPLGQLVQTGTCVHVEGLLKVPPEGTKQRVELRVEKVHHVGPVDPAKYPLPKTRLTLEFLRDFVHFRPRTNTISAVARIRNALAYATHTFFQNHGFLYIHTPIITTSDCEGAGEMFQVTTLISDAEKVEKELIKNPPPSEADIEAAKALVKEKGEAVAQLKSAKASKGEITASVAELNKAKENLSRLEERSKLKPGIPQKDGKIDYSQDFFARQAFLTVSGQLQVETYACAVSSVYTFGPTFRAEHSHTSRHLAEFWMVEPEIAFADLKDDMNCAEAYVKFLCQWLLDNCIDDMEFMAKNFDKGCIDRLRMVASTPFERISYTEAIKLLEEAVKKDKKFENKVEWGIDLASEHERFLTEVLFKKPVIVHDYPKGIKAFYMRLNDDMKTVAAMDVLVPKVGELIGGSQREERYEVIEKRILEMGLPLEPYEWYLDLRRYGTVKHCGFGLGFERMILFATGIDNIRDVIPFPRYPGRADL

SEQ ID NO: 6 - protein, 569 aa, Malus domestica
MADDSTPPSAQLAAVTLSNDASVLKAEFSDRVLIRSIISRPDGGSGLAGQKIRIGGWVKTGRKADKDAFAFLELNDGSCASNLQVIVKADKYDLGQLVQTGTCLSVDGVLKLPPDGKKQKVELDVEKVIHVGPVDPAKYPLPKTRLTLEFLREHVHLRSRTNTISAVTRIRNALAFATHSFFHKHGFLYVQTPIITTSDCEGAGEMFQVTTLFSEAEKLEKDLIENPPPSEADIEAAKLIVKERGDDVAQLKSAKASKQEIGAAVAELKKAKDNVLKLEERSKLKPGIPRKDGKVDYTQDFFARQAFLTVSGQLQVESYACALSSVYTFGPTFRAENSHTSRHLAEFWMVEPEIAFAELEDDMNCAEAYVKFLCQWLLDDCREDMEFMADKIDKTCIDRLTMVAKTPFKRITYTEAVDLLIDAVKKGKKFENHVEWGIDLASEHERYLTEVLFQKPVIVYNYPKGIKAFYMRLNDDLKTVAAMDVLVPKVGELIGGSQREERYDIIMSRIEEMGLPAEPYEWYLDLRRFGTVKHCGFGLGFERMVLFATGIDNIRDVIPFPRYPGRADL

SEQ ID NO: 7 - protein, 567 aa, Citrus sinensis
MADNELPVDQVATMDLNDDAVQRHQFSDRVLIKSILTRPDGGAGLAGRQVRVGGWVKTGREQGKGSFAFLEVNDGSCPANLQVIVDKDVADLGQLVPTGTCVYVEGMLKNPPEGTKQKIELRVQKVVDVGMVDPAKYPIPKTKLTLEFLRDRIPFRPRTNTIAAVARIRNALAYATHTFLQKQGFLYIHTPIITTSDCEGAGEMFQVTTLISDADKLEKELIKNPPPSEADIEAAKLVIKEKGEAVAKLKSDKAGREAISASVTELTKAKENLAKLEERSKLKPGIPQKDGKIDYTQDFFARQAFLTVSGQLQVETYACAVSNVYTFGPTFRAEHSHTSRHLAEFWMVEPEMAFSDLKDDMNCAEAYVKFMCDWLLDHCFDDMEFMAKNYDKSCINRLRMVASTPFERITYTEAVELLEVAVKEGKHFENKVEWGIDLASEHERYLTEVKFQKPVIVYNYPKGIKAFYMRLNDDLKTVAAMDVLVPKVGELIGGSQREERYDVIKSRIEDMGLPLEPYEWYLDLRRFGTVKHSGFGLGFERMILFATGIDNIRDVIPFPRYPGRADL

SEQ ID NO: 8 - protein, 566 aa, Glycine max
MADASSPPTDQLAAITLDEVPKANFSDRVPIRSIISRTDGGSGLAGRKARVGGWVKTGRKADKDAFAFLEINDGSCAGNLQVIVEAALGELGQLVPTGTCVVVDGHLKLPPAGTRQKIELRADKVLHVGPVDPAKYPLPKMRLTLEFLRDFVHLRSRTNTISAVARIRNALAYATHTFFNKEGFLYVHTPIVTTSDCEGAGEMFQVTTLLSEAERLEKELLQNPPPSEADVEAARVVVQEKGEVVSQLKAAKASKQEIGAAVDQLKKAKESLAKVEEWSKLKPGIPKKDGKVDYGKDFFARQAFLTVSGQLQVESYACALSSVYTFGPTFRAENSHTSRHLAEFWMVEPEIAFAELKDDMNCAEAYVKFMCQWLLDNCLEDMEFMADKFDKGCIDRLKLVASTPFIRVTYTEAVEILEDAVKNGKKFENEVKWGIDLASEHERYLTEVKFQKPVIVYNYPKDIKAFYMRLNDDLKTVAAMDVLVPKVGELIGGSQREERYDVIQTRIKEMGLPVEPYEWYLDLRRYGTVKHAGFGLGFERMILFATGLENIRDVIPFPRYPGRADL

SEQ ID NO: 9 - protein, 561 aa, Arachis hypogaea
AEQLAATTINDDGIVPKAEFSDRVPINSIISRADGGSGLAGKKARVGGWVKTGRKADKDAFAFLELNDGSCPGNLQVIVEAGLVELSQVVPTGTCVVLDGELKLPPEGAKQKVELRAEKVVHVGPVDPAKYPLPKMRLTLEFLRDFVHLRSRTNTISAVARIRNALAYATHTFFNKHGFLYVHTPIITTSDCEGAGEMFQVTTLLSEAERLEKELIHNPPPTEAEVEAARVVVKEKGEIVSQLKSAKASKKEIGAAVDELKKSKDTLSKLEERSKLKPGIPKKDGNVDYAKDFFARQAFLTVSGQLQVESYACALSSVYTFGPTFRAENSHTSRHLAEFWMVEPELAFAELKDDMNCAEAYVKFLCQWLLDNCLEDMKFMADKFDKGCIDRLKMVASTPFVRVSYTEAVEILEEAVKNGKKFDNEVKWGIDLASEHERYLTEVKFQKPVIVYNYPKDIKAFYMRLNDDSKTVAAMDVLVPKVGELIGGSQREERYDVIVQRIKEMGLPLEPYEWYLDLRRYGTVKHSGFGLGFERMILFATGLENIRDVIPFPRYPGRADL

SEQ ID NO: 10 - protein, 565 aa, Prunus dulcis
MADDLTAQLATATLDNNASVQRAQFSNRVPIKSIILRPDGGSGLAGKLARVGGWVKTGRKADKDAFAFLELNDGSCSGNLQVIVEADKGDLGQLVLTGTSLVVDGVLKLPPDGKRQKVELRVEKVIHVGLVDPSKYPLPKTRLPLEFLRDYVHLRSRTNTISAVLRIRDALAYATHTFFHDHGFRYVQTPIITTSDCEGAGEMFQVTTLINEAEKLEKDLIENPPSEADIEAAKLIAKERGIDVAQLKSAKASKQEIGAAVVELQKAKDNLVKLEERSKLQPGIPRKDGKIDYTQDFFGRQAFLTVSGQLQVESYACSLSTVYTFGPTFRAENSHTSRHLAEFWMVEPEIAFAELEDDMNCAEAYVKYLCQSLLDNCREDMEFMADKIDKSCIDRLTMVAKTPFVRITYTEAVELLTEAVKNGKKFENHVEWGIDLASEHERYLTEVRFQKPVIVYNYPKGIKAFYMRLNDDSKTVAAMDVLVPKVGELIGGSQREERYDVIVSRIKEMGLPLEPYEWYLDLRRYGTVKHCGFGLGFERMVLFATGIDNIRDVIPFPRYPGRADL

SEQ ID NO: 11 - protein, 566 aa, Lactuca sativa
MADNGVLPVDKLTISETVKEAEFSQRVLVRSILGRPDGGAGLAGQTLKVGGWVKKGREQGKGSFAFLELNDGSCTANLQLIIYSDVAPLGQFTPTGTSLHVEGVLQMPPADKQGKQSIELKVSKVLDVGAADPAKYPLPKTRLTLEFLRDYVHLRPRTNTISAIARIRNALAYATHTFFQKHGFLYVHTPIITTSDCEGAGEMFQVTTLINDSEKLEKELLKNPPPSQEDVDAARAAVKEKGGIVAKLKSDKADKSAITVAVAELTKAKETLSKIEERFNQKPGIPKKDGKVDYSQDFFARQAFLTVSGQLQVETFACALSSVYTFGPTFRAEHSHTSRHLAEFWMVEPEIAFADIEDDMKCAEAYVRFMCQWLLDNCLDDMEFIAEKFDEHAINRLKMVASTNFVRLTYTEAVTILEEAVSKGHQFENKVEWGIDLASEHEKYLTETKFESPVIVYNYPKGIKAFYMKVNPDNKTVAAMDVLVPKVGELIGGSQREENYEVIKERILEMGLPLEPYEWYLDLRRYGTVKHSGFGLGFERMILFATGIDNIRDVIPFPRFPGRADL

SEQ ID NO: 12 - protein, 569 aa, Fragaria vesca
MADDAAQLGSQLAETALNESVSDLKAEFSDRVPIKSIISRPDGGSAFAGQKVRVGGWVKTGRKADKDAFAFLELNDGSCPGNLQVIVQADKGDLGQLVPTGTCVVVEGELKLPPAGVKQKVELRVEEVLHLGPVDPLKYPLHKGKISLERLRDVVHLRPRTNTISAVARIRDDLAYATHTFFRKHGFRYVHTPIITTSDCEGAGEMFQVTTLISEGERLERELIKNPPPSEADVEAANLIVTEKGDAVSQLKSAKASKDEIGAAVAELKRAKENVLKLQERAKLQPGIPKKDGKVDYTQDFFARQAFLTVSGQLQVESYACAISSVYTFGPTFRAENSHTSRHLAEFWMVEPELAFAELKDDMNCAEAYVKFLCKWLLDNCYDDMEFFSRQYDKTCIERLRMVASTPFERITYTEAVELLIDAVKNGKKFENPVEWGIDLASEHERFLTEVKFQKPVIVYNYPKGIKAFYMRLNDDNKTVAAMDVLVPKVGELIGGSQREERYDVIHSRIAEMDLPIEPYEWYLDLRRFGTAKHSGFGLGFERMVLFATGMDNIRDVIPFPRFPGRADL

SEQ ID NO: 13 - protein, 563 aa, Solanum tuberosum
MSVDSAPPVEKLTLSDAVEEAKFSQRVPIRSIVGRPDGGAGLAGNVVKIGGWVKTGREQGKGSFAFLEVNDGSCPGNLQVIVDASVHKLGDLVLTGTCVHVEGELKVPPEGAKQKVELRVQKVISVGTVDAAKYPLPKTKLTLEFLRDVVHLRSRTNTISAVARIRNALAYATHTFFQNNGFLYIHTPIITTSDCEGAGEMFQVTSLISEAEKLEKELKENPAPSESDIQAAEQLAKEKGEVVAQLKASKASNKKISAAVAELQRAKENLLKLQERSRLSAGIPKKDGKIDYSEDFFGRQAFLTVSGQLQVETYACALSSVYTFGPTFRAEQSHTTRHLAEFWMVEPEIAFADIQDDMNCAEAYVKFLCQWLLDHCLDDMEFMTKFVDKDALSRLRMVASSNCHRITYTEAIDILEVAAKVKKFENKVEWGIDLASEHERYLTEEHFKAPVIVFNYPKGIKAFYMKVNEDNKTVAAMDLLVPKVGELIGGSQREENYEVLRSRILDMGLPLEPYEWYLDLRRYGTVKHSGFGLGFERMILFATGIENIRDVIPFPRYPGRADL

SEQ ID NO: 14 - protein, 563 aa, Solanum lycopersicum
MSVDSAPPVEKLTLTDVVEEARFSQRVPICSIVGRTDGGAGLAGNVVKIGGWVKTGREQGKGSFAFLEVNDGSCPGNLQVIVDASVHKLGDLVLTGTCVHVEGELKVPPEGAKQKVELRVQKVISVGTVDAAKYPLPKTKLTLEFLRDVVHLRSRTNTISAVARIRNALAYATHTFFQNNGFLYIHTPIITTSDCEGAGEMFQVTSLISEAEKLEKELKENPAPSESDTQAAEKLAKEKGEVVAQLKASKASNKKISAAVAELQRAKENLLKLQERSRLSAGIPKKDGKIDYSEDFFGRQAFLTVSGQLQVETYACALSSVYTFGPTFRAEQSHTTRHLAEFWMVEPEIAFADIQDDMNCAEAYVKFLCQWLLDHCLDDMEFMTKFVDKDALSRLRMVASSNCHRITYTEAIDILEVAAKVKKFENKVEWGIDLASEHERYLTEEHFKAPVIVFNYPKGIKAFYMKVNEDNKTVAAMDLLVPKVGELIGGSQREENYEVLRSRILDLGLPLEPYEWYLDLRRYGTVKHSGFGLGFERMILFATGIENIRDVIPFPRYPGRADL

SEQ ID NO: 15 - protein, 536 aa, Oryza sativa
TDRTRIRSILADGAARAGERVVVGGWVKTGREQGKGTFAFLELNDGSCASNLQVLVDAAVHPLAPLTATGTSVLVEGELKKPPEGAKQRVELRVDRVIEVGEVDPAAYPLPKTKLTLENLRDVVHLRSRTNTIGAVARIRHQLACATHRFFDENGFLYVHTPIITTSDCEGAGEMFQVTTLFSHAEKVEKELKENPAPSESDIEAARVVVKEKGDAVAQLKAAKASKQEITAAVAELNKAKENVSRLEERSKLKPGIPRDDGTVAYENDFFKRQAFLTVSGQLQVETYACALSSVYTFGPTFRAENSHTSRHLAEFWMVEPEIAFANLQDDMNCAERYVQYLCKWLLEHCREDMEFMVKNYDKTAIERLELVSSTPFQRISYTKAVELLKNVTDKKFENKVEWGIDLASEHERYLTEVIFKKPVIVYNYPKEIKAFYMRLNDDQKTVAAMDVLVPKVGELVGGSQREERLDLLKTRIQDAGLPLEPYEWYLDLRRFGSVKHSGFGLGFERMILFATGLENIRDVIPFPRYPGRADL

SEQ ID NO: 16 - protein, 553 aa, Ananas comosus
GDESAPPTSEMAEPSLQRTLIKSIVSSSAAAAALVGRRVVVGGWVKTGREQGKGTFAFLELNDGSCLANLQVIVDAKVHPLSELVPTGTCVLVEGELKKPPEGTKQRIELRVDKVLALGPVDSKYPLPKTRLTLENLREVVHLRARTNTISAVARIRDELAYGTHTFFRKNGFRYVHTPIITTSDCEGAGEMFQVTTLFSLAEKTEKELKQNPPPSESEVEAAKVLVKEKGEAVAQLKSSKASKEEISASVAELTKAKENLSRLEERFKLKPGIPRKDGGIAFENDFFKRQAFLTVSGQLQVETYACALGNVYTFGPTFRAENSHTSRHLAEFWMVEPEMAFANLQDDMNYAESYVKFLCQWLLDHCLEDMEFMVKNYDKTAIERLKLVASVPFEHISYTKAVQLLKNVTDKEFENKVEWGIDLASEHERYLTEVIFKKPVIVYNYPKGIKAFYMRLNDDQKTVAAMDVLVPKVGELIGGSQREERIDVLKSRILESGLPLEPYEWYLDLRRYGTVKHCGFGLGFERMIMFATGLDNIRDVIPFPRYPGKADL

SEQ ID NO: 17 - protein, 535 aa, Triticum aestivum
TSRTRIRAILDAGDAMAGERVVVGGWVKTGRQQGKGEFAFLEVNDGSCQGNLQVMVDKDVHPLASLTHTGTSVLVEGVLKKPPAEAKQRIELKVERVIELGEVDAAAYPLPKTKITLETLRDFVHLRARTNTIGAVARIRHQLAYATHSFFDENGFLYIHTPIITTSDCEGAGEMFQVTALFSQAEKVEKELKENPAPSEADVEAAKLVVKEKGDAVAQLKAAKASKQEITAAVSVLTKAKENVLRVEERSKLKPGLPKDDGKIAFENDFFKRQAFLTVSGQLQVETYACALSNVYTFGPTFRAENSHTSRHLAEFWMVEPEIAFANLQDDMNCAERYVQYLCKWLLKHCREDMEFMVKHVDKTAIERLELVSSTPFERISYTKAVEILEGVDKKFENKVEWGIDLASEHERYLTEVIFKKPVIVYNYPKGIKAFYMRLNDDQKTVAAMDVLVPKVGELIGGSQREERLDILKQRILDADLPLEPYEWYLDLRRFGSVKHSGFGLGFERMILFATGLENIRDVIPFPRYPGRADL

SEQ ID NO: 18 - protein, 516 aa, Zea mays
VVVGGWVRTGREQGKGSFAFLELSDGSCAATLQAIVDAAVHPLARLTATGTSVLVEGVIKEPPEGTKQNVELKVSRVLEVGEVDAAVYPLPKVKLTLEKLRDVVHLRSRTNTIGAVARIRHQLAYASHRFFDENGFLYVHTPIITTSDCEGAGEMFQVTTLFSQAEKTEKELRENPKPSDSEIEAAKVLVKEKGDVVAQLKAAKASKQEISTAVDELNRAKEIVSKLEERFKLKPGIPRKDGSIAFENDFFKRQAFLTVSGQLQVETYACALSSVYTFGPTFRAENSHTSRHLAEFWMVEPEIAFANLQDDMNCAEKYVQYLCKWLLDHCQEDMEFMVKNYDKSAIERLELVSSTPFVRISYTKAVELLKNVTDKKFDNKVEWGIDLASEHERYLTEDIFKKPVIVYNYPKGIKAFYMRLNDDDKTVAAMDVLVPKVGELIGGSQREERLDVLKQRILDAGLPLEPYEWYLDLRRFGSVKHSGFGLGFERMILFATGMENIRDVIPFPRYPGRADL

SEQ ID NO: 19 - protein, 544 aa, Beta vulgaris
APFSKRIPIRSILNRSDGGASLAGQRVTVRGWVRTGRKQNKGALGFIELNDGSCCANLQVIVESSVCELSQLLSPGSCVSLSGELKSPPQSGNDKQKIELFADHVIEVVPVDPTEYPLPKTRIITEYLRDFVHLRPRTSSFAAVARIRSELAYATHTFFRDNGFINVHTPIITTIDCEGAGETFQVTTLIKDAEKLEQELLENPAPSEADVEAAKLVVEEKGEVLKKLKAAEASKEEIKAAVGVLMKAKGDLAKLEERAKLQPGIPKKDGKVDYAHDFFARQAFLTVSGQLQAESFACSLGSVYTFGPTFRAEHSHTSRHLAEFWMVEPEIAFADLEDDMNCAEAYIKFMCQWLLDRCLDDMVVIAKLFDNTAIDRLKMVASTAFVRITYTEAVTLLEQVKDKKFENKVEWGSDLASEHERYLTEVIFKKPVIVYNYPKGIKAFYMRLNDDGKTVAAMDVLVPKVGELVGGSQREERYDVIVKRIVEMGLPLEHYKWYLDLRRYGTAIHAGFGLGFERMLLFATGIDNIRDVIPFPRYPGRADL

SEQ ID NO: 20 - DNA, 22 bp, synthetic construct
  source 1..22: mol_type = other DNA
caccatggctgatgagattgtg

SEQ ID NO: 21 - DNA, 24 bp, synthetic construct
  source 1..24: mol_type = other DNA
aagatcagcttttccaggatagcg

SEQ ID NO: 22 - DNA, 11 bp, synthetic construct
  source 1..11: mol_type = other DNA
gctttgagacc

SEQ ID NO: 23 - DNA, 2014 bp, synthetic construct
  source 1..2014: mol_type = other DNA
  misc_feature 1..8: note = 5' adaptor
  misc_feature 2004..2014: note = 3' adaptor
ggtctctaatgtacccgtacgacgtacctgattatgctgccggggcctacccctacgatgtgccagactatgcggcgggagcatatccatatgatgtacccgactacgcaggtggtggatccggtggggggtccgggggttcggcgtggagccacccgcagtttgaaaaaggtggcgggagcggaggcggcagcggcgggtccgcctggtcacatcctcagttcgagaaatcaggctctgagacacccggcacttcagaatcagccactccagagtccgggtctatggctgacgagattgttccgccagctactcaactcgcggcagtgagcctcgaaaatgacgggtctacagtacagcgcgctcaattcagtaacagagtgctgatccgtaccattttagatcggccagatggtggcgcgggccttgctgggcagactgtccggattggaggatgggtcaaatccgggcgggaccaaggcaagaggacattttctttcctggcggtgaatgacggaagttgtcctgctaatctgcaggtgatggttgatccatcgctctatgacgtgagcaatttggtagctacgggcacctgtgtcaccgtcgatggtgtgcttaaggttccgcctaaaggcaaggggacccaacagcagatcgagcttaatgttgtcaaagtgattgatgttggaacggtggacgcctcaaagtatcctctgcctaagaccaagctgacgttagagactctgagggacgtgctccacctaaggagcaggactaatagcatttctgctgttgcaaggatacgtaatgctctcgccttcgccactcatagcttcttccaggagcacagtttcctgtacattcatacgccgatcatcaccacctcggactgcgagggtgcgggtgagatgtttcaagcgaccactcttatcaactacaccgagcggctggagcaagatcttatagacaaccccccgccgactgaagcagacgtggaggcagcgagactgatcgtcattgagagaggaaacgtggtggctgaattgaaagcagcaaaggcgagcaaggaggctatcacggcggcagtagctgagctgaagatcgccaaagaaaccttcgcgcacattgatgaacgctcacgattgaggcctggactacccaagaaggacggcaacatagattactccaaggattttttcggacgtcaggcctttctaacagttagtggtcagttgcaggttgaaacatacgcctgtgccctctccaatgtgtacactttcggcccgacgttccgcgcagaaaattcccatacatcgcgccacctggccgaattctggatggtcgagcctgaaattgcctttgctgatttggaagatgacatgaactgcgcggaggcttacgtgaagtacatgtgcaactggttgctagagaagtgctatgcagatatggagttgatggcgaagaacttcgacagtggctgcatcgacaggcttaagttagtagcttcaaccccctttggccgcataacatatacaaaggccatcgagcttcttgaggaagccgtcgccaaggggaaagagtttgataacaacgttgaatgggggattgacctcgcgtctgagcatgaaagatatttgacagaagtgcttttccaaaagccactcatcgtgtacaactatcccaaaggcatcaaggcgttctacatgcgcctgaatgatgatgagaaaacagtcgccgccatggacgtcctcgtcccaaaagttggcgaactcataggtggaagccagagagaggagcgctatgatgtcatcaaaaagagaattgaagagatgggcctacccatagaaccatacgagtggtacctggatctcaggaggtatggaacagtcaagcactgcggctttggattgggctttgagcgcatgattttgtttgcaacgggcctcgacaacatacgggatgttatcccattcccgcggtaccctggcaaagctgacctgtgagctttgagacc

SEQ ID NO: 24 - DNA, 2014 bp, Zea mays
  source 1..2014: mol_type = genomic DNA
ggtctcaggagctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagaatgagagacc

SEQ ID NO: 25 - DNA, 932 bp, Zea mays
  source 1..932: mol_type = genomic DNA
ggtctcagcttgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgactcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgaccgcttgagacc

SEQ ID NO: 26 - DNA, 1341 bp, Cauliflower mosaic virus
  source 1..1341: mol_type = genomic DNA
atctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggaca

SEQ ID NO: 27 - DNA, 346 bp, Cauliflower mosaic virus
  source 1..346: mol_type = genomic DNA
tgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggaca